(19) United States
(12) Patent Application Publication
(10) Pub. No.: US 2012/0245099 A1
(43) Pub. Date: Sep. 27, 2012

(54) APOPTOSIS INDUCER

(75) Inventors: Chiaki Hidai, Chiyoda-ku (JP); Hisataka Kitano, Chiyoda-ku (JP)

(73) Assignee: NIHON UNIVERSITY, Tokyo (JP)

(21) Appl. No.: 13/392,061

(22) PCT Filed: Aug. 25, 2010
(86) PCT No.: PCT/JP2010/064857

§ 371 Date: Mar. 9, 2012

(30) Foreign Application Priority Data
Aug. 25, 2009 (JP) 2009-194747

(51) Int. Cl. A61K 38/10; A61P 35/00; C07K 14/47; C12N 5/09; C07K 7/08

(52) U.S. Cl. 514/19.3; 435/375; 530/327; 530/350; 530/324

(57) ABSTRACT

The present invention provides an apoptosis-inducing agent capable of inducing apoptosis in various cells including tumor cells and other cells in a simple manner, etc. The apoptosis-inducing agent of the present invention comprises a peptide shown in (a) or (b) below, a derivative thereof, or a salt of the peptide or the derivative: (a) a peptide which comprises an amino acid sequence represented by the formula: C—X-D-X—X—X—X—Y—X—C—X—C (SEQ ID NO: 1)(I); or (b) a peptide which comprises an amino acid sequence with deletion, substitution or addition of one or several amino acids in the amino acid sequence represented by formula (I) and which has apoptosis-inducing activity.

### TECHNICAL FIELD

The present invention relates to apoptosis-inducing agents, etc. More specifically, the present invention relates to apoptosis-inducing agents which induce apoptosis in various cells including tumor cells and other cells, etc.

### BACKGROUND ART

Apoptosis is one of the modes of cell death and particularly refers to cell death associated with nuclear destruction in cells. Molecular mechanisms previously known for apoptotic death in cancer cells are those induced thorough the following five pathways: calcium pathway, death signaling pathway, ceramide pathway, mitochondrial pathway and p53 apoptosis pathway (see, e.g., “Yoshiyuki Hashimoto, New Molecular Medicine of Apoptosis, Yodosha Co., Ltd., Japan, April 2001, pages 10-58”).

Many substances are known to induce apoptosis, and anticancer agents such as mitomycin and taxol are used in clinical medicine. In addition to these artificial substances, apoptosis-inducing proteins such as FasL (Fas ligand) and TNFα can be found in vivo. These artificial and natural apoptosis-inducing substances can be used to induce the death of cells which are harmful to the human body, such as cancer cells. As described above, not only anticancer agents used in chemotherapy, but also FasL which is now being studied for its use in gene therapy relies on this principle. In chemotherapy, side effects on normal tissues serve as a hindrance to the therapy, and hence studies have now been conducted on gene therapy involving gene transfer into a lesion, with the expectation of producing an effect only on the lesion. However, cells that will actually receive gene transfer are usually limited to some of the cells present in a lesion. Moreover, diseased cells receiving gene transfer will then die and therapeutically effective proteins or the like will be no longer produced from these cells. Eventually, the effect of gene therapy is local in most cases. For this reason, residual diseased cells are required to repeatedly receive gene transfer. Moreover, cells receiving no gene during the fist gene transfer may be resistant to gene transfer and serve as a hindrance to the therapy.

### SUMMARY OF THE INVENTION

Under these circumstances, there has been a demand for the development of apoptosis-inducing agents capable of inducing apoptosis in various cells including tumor cells and other cells in a simple manner.

The present invention has been made in consideration of the above situation and aims to provide an apoptosis-inducing agent, a pharmaceutical composition for cancer treatment and so on, as shown below.
(1) An apoptosis-inducing agent, which comprises a peptide shown in (a) or (b) below, a derivative thereof, or a salt of the peptide or the derivative:
(a) a peptide which comprises an amino acid sequence represented by the following formula (I):

(I)
(SEQ ID NO: 1)
C-X-D-X-X-X-X-Y-X-C-X-C

(wherein X represents any amino acid residue, C represents cysteine, D represents aspartic acid, and F represents phenylalanine); or
(b) a peptide which comprises an amino acid sequence with deletion, substitution or addition of one or several amino acids in the amino acid sequence represented by formula (I) and which has apoptosis-inducing activity.

In the inducing agent according to (1) above, the amino acid sequence represented by formula (I) may be, for example, the amino acid sequence shown in SEQ ID NO: 2 or SEQ ID NO: 3. Likewise, the peptide shown in (a) above may be, for example, a peptide comprising the amino acid sequence shown in SEQ ID NO: 5 or SEQ ID NO: 7.
(2) A method for inducing apoptosis, which comprises the step of treating a target cell or a target cell population with the inducing agent according to (1) above.

In this method, the above step may be, for example, intended to contact the target cell or target cell population with the inducing agent.
(3) A pharmaceutical composition for cancer treatment, which comprises the inducing agent according to (1) above.

### BRIEF DESCRIPTION OF THE DRAWINGS

FIG. 1 shows the results observed under a phase contrast microscope and a fluorescent microscope in Example 1.

FIG. 2 shows the results of polyacrylamide gel electrophoresis (left panel) and the results measured for lactate dehydrogenase (LDH) activity (right panel) in Example 2.

FIG. 3 shows the results observed under a fluorescent microscope in Example 3.

FIG. 4 shows the results observed under a fluorescent microscope in Example 4.

FIG. 5 shows the results observed under a fluorescent microscope in Example 5.

FIG. 6 shows the results of polyacrylamide gel electrophoresis and silver staining in Example 6.

FIG. 7 shows the results measured for LDH activity in Example 7.

FIG. 8 shows the results observed under a phase contrast microscope and a fluorescent microscope in Example 8.

FIG. 9 shows the results measured for LDH activity in Example 9.

FIG. 10 shows the results measured for LDH activity in Example 10.

FIG. 11 shows the results measured for LDH activity in Example 11.

FIG. 12 shows the results of tumor size after each gene transfer, as measured over time (for 7 days: Day0 to Day7) in Example 12. For measurement of tumor size, the long axis and short axis of each tumor were measured with a ruler and multiplied by each other. In the figure, the tumor size (vertical axis) is expressed as a relative value (mean±SD), assuming that the value obtained at the time of gene transfer is set to 1.

### BEST MODES FOR CARRYING OUT THE INVENTION

The present invention will be described in more detail below. The scope of the present invention is not limited by the following description, and any embodiments other than those illustrated below may also be carried out with appropriate modifications without departing from the spirit of the invention.

It should be noted that this specification incorporates the specification of Japanese Patent Application No. 2009-194747 (filed on Aug. 25, 2009) in its entirety, based on which the present application claims priority. Moreover, all publications cited herein, including prior art documents, patent gazettes and other patent documents, are incorporated herein by reference.

### 1. Apoptosis-Inducing Agent

As described above, the apoptosis-inducing agent of the present invention (hereinafter referred to as the inducing agent of the present invention) comprises a peptide shown in (a) below:
(a) a peptide which comprises an amino acid sequence represented by the following formula (I):

(I)
(SEQ ID NO: 1)
C-X-D-X-X-X-X-Y-X-C-X-C.

In the peptide shown in (a) above, the amino acid sequence of formula (I) is expressed with one letter codes of amino acids. For example, C, D and Y denote cysteine (Cys), aspartic acid (Asp) and tyrosine (Tyr), respectively. X represents any amino acid residue, which may generally be selected from 20 types of amino acid residues, i.e., A (alanine; Ala), R (arginine; Arg), D (aspartic acid; Asp), N (asparagine; Asn), C (cysteine; Cys), Q (glutamine; Gln), E (glutamic acid; Glu), G (glycine; Gly), H (histidine; His), I (isoleucine; Ile), L (leucine; Leu), K (lysine; Lys), M (methionine; Met), F (phenylalanine; Phe), P (proline; Pro), S (serine; Ser), T (threonine; Thr), W (tryptophan; Trp), Y (tyrosine; Tyr) and V (valine; Val). In this specification, other amino acid sequences may also be expressed with one letter codes, as in the case of formula (I).

Preferred examples of the above amino acid sequence of formula (I) include the following amino acid sequences shown in SEQ ID NO: 2 and SEQ ID NO: 3. Among them, more preferred is the amino acid sequence shown in SEQ ID NO: 2.

(SEQ ID NO: 2)
C-T-D-L-V-A-N-Y-S-C-E-C
(SEQ ID NO: 3)
C-K-D-D-I-S-S-Y-E-C-W-C

As used herein, the term “peptide” refers to a structure in which at least two amino acids are linked together via peptide bonds, including oligopeptides, polypeptides and so on. Moreover, a polypeptide formed into a certain three-dimensional structure is referred to as a protein, and such a protein also falls within the scope of the above “peptide” in the present invention. Thus, the peptide included in the inducing agent of the present invention may be any of an oligopeptide, a polypeptide and a protein.

Alternatively, the inducing agent of the present invention may comprise a peptide shown in (b) below, as described above:
(b) a peptide which comprises an amino acid sequence with deletion, substitution or addition of one or several amino acids in the amino acid sequence represented by formula (I) above and which has apoptosis-inducing activity.

Examples of the above “amino acid sequence with deletion, substitution or addition of one or several amino acids” include, but are not limited to, amino acid sequences with deletion, substitution or addition of about 1 to 5 amino acids, preferably about 1 to 2 amino acids. However, preferred for use in the present invention are those without deletion, substitution or addition of amino acid residues at positions 1, 3, 8, 10 and 12 (C, D, Y, C and C, respectively, in this order) in the amino acid sequence represented by formula (I) above, and/or those without deletion or addition of amino acid residues at positions 2, 4-7, 9 and 11 (each of which is X) in the amino acid sequence represented by formula (I) above. Introduction of mutations such as deletion, substitution or addition as described above may be accomplished by using a kit for mutation introduction based on site-directed mutagenesis, including GeneTailor™ Site-Directed Mutagenesis Systems (Invitrogen) and TaKaRa Site-Directed Mutagenesis Systems (e.g., Mutan-K, Mutan-Super Express Km; Takara Bio Inc., Japan). Further, whether or not a peptide has the above mutation (i.e., deletion, substitution or addition) can be confirmed by using various techniques for amino acid sequencing, as well as X-ray or NMR-based structural analysis, etc.

As used herein, the term “apoptosis-inducing activity” is intended to mean the ability to induce cell death associated with nuclear destruction in cells, and this activity can be measured, for example, by DNA ladder detection, chromatin staining, annexin V staining, etc.

The above peptide (a) or (b) included in the inducing agent of the present invention may be constructed from any number of amino acid residues as long as it is a peptide comprising the above amino acid sequence of formula (I) or a peptide comprising an amino acid sequence with deletion, substitution or addition as described above. The number of amino acid residues is preferably 12 or more, and may be 12 to 100, 12 to 50, or 12 to 30, by way of example.

The above peptide (a) or (b) may be either derived from a natural product or artificially obtained by chemical synthesis. Without being limited thereto, preferred are peptides derived from natural products because they often have no adverse effects (e.g., cytotoxicity) on any cells other than target cells of apoptosis induction.

Examples of peptides derived from natural products include naturally-occurring oligopeptides, polypeptides and proteins, or fragments thereof, etc. Such a peptide derived from a natural product may be obtained directly from the natural product by known collection and purification techniques or may be obtained by known gene recombination technology, in which a gene encoding this peptide is integrated into any of various expression vectors or the like and introduced into a cell to express the peptide, followed by known collection and purification techniques. Alternatively, such a peptide may be produced in a cell-free protein synthesis system using a commercially available kit (e.g., a reagent kit PROTEIOS™ (Toyobo Co., Ltd., Japan) or TNT™ System (Promega), a synthesizer PG-Mate™ (Toyobo Co., Ltd., Japan) or RTS (Roche Diagnostics)) and obtained by known collection and purification techniques. Peptides derived from natural products may be obtained in any way.

On the other hand, chemically synthesized peptides can be obtained by using known peptide synthesis techniques. Examples of synthesis techniques used for this purpose include azide method, acid chloride method, acid anhydride method, mixed acid anhydride method, DCC method, active ester method, carboimidazole method and oxidation-reduction method, etc. Moreover, their synthesis may be accomplished by applying either solid phase synthesis or liquid phase synthesis. A commercially available peptide synthesizer may also be used for this purpose. After synthesis reaction, the resulting peptides may be purified by using known purification techniques (e.g., chromatography) in combination.

As examples of a peptide derived from a natural product and falling within the above peptide (a) or (b), (i) developmentally regulated endothelial cell locus-1 (Del-1; developmentally endothelial locus-1) protein and a partial fragment thereof, as well as (ii) blood coagulation factor IX will be illustrated below.

Del-1 protein is an extracellular matrix protein having epidermal growth factor (EGF)-like domains and discoidin I-like domains, which consists of the amino acid sequence shown in SEQ ID NO: 5. This Del-1 protein is known to have EGF1, EGF2 and EGF3 as EGF-like domains and have Discoidin1 and Discoidin2 as discoidin I-like domains. The inventors of the present invention have focused on EGF3 among the above various domains and have further focused on a region consisting of specific 12 amino acid residues: C-T-D-L-V-A-N—Y—S—C-E-C (SEQ ID NO: 2) in the amino acid sequence constituting EGF3 (SEQ ID NO: 7). As a result, the inventors have found that a peptide consisting of this specific region or comprising this specific region has high apoptosis-inducing activity. Namely, examples of a peptide derived from a natural product and falling within the above peptide (a) or (b) include the full-length Del-1 protein (SEQ ID NO: 5), a partial fragment consisting of EGF3 (SEQ ID NO: 7), and various partial fragments comprising EGF3 (SEQ ID NOs: 9, 11, 13, 15, 17 and 19 (see Table 1 below), as well as SEQ ID NO: 42). In particular, preferred for use as the above peptide (a) is the full-length Del-1 protein (SEQ ID NO: 5), a partial fragment consisting of EGF3 (a peptide constituting EGF3; SEQ ID NO: 7), or a fusion peptide between EGF3 and Discoidin1 (SEQ ID NO: 42). For details of the Del-1 protein and partial fragments thereof, reference may be made to the entire contents disclosed in WO2005/0001093. In addition to partial fragments having the amino acid sequences shown in Table 1 below, other partial fragments may also be included as embodiments of the present invention. By way of example, a partial fragment having an amino acid sequence modified to remove the signal peptide segment from “full-length Del-1” in Table 1 below is also included as an embodiment of the present invention.

TABLE 1

| Name of fragment | | | |
|---|---|---|---|
| or the like | Region* | Type | SEQ ID NO: |
| Full-length Del-1 | 619-2061 | DNA | 4 |
| | 1-480 | Protein | 5 |
| EGF3 | 985-1089 | DNA | 6 |
| | 123-157 | Protein | 7 |
| 4-1 | 619-1662 | DNA | 8 |
| | 1-348 | Protein | 9 |
| 4-15 | 619-1713 | DNA | 10 |
| | 1-365 | Protein | 11 |
| 4-14 | 619-1722 | DNA | 12 |
| | 1-368 | Protein | 13 |
| 4-13 | 619-1773 | DNA | 14 |
| | 1-385 | Protein | 15 |
| XY | 985-1662 | DNA | 16 |
| | 123-348 | Protein | 17 |
| XC | 985-1269 | DNA | 18 |
| | 123-217 | Protein | 19 |
| human XY | — | DNA | 20 |
| | — | Protein | 21 |

*Regions in DNAs are indicated by nucleotide numbers, while regions in proteins are indicated by amino acid numbers.
It should be noted that nucleotide numbers and amino acid numbers used herein are corresponding numbers on the full-length Del-1 sequence.

Blood coagulation factor IX, which consists of the amino acid sequence shown in SEQ ID NO: 23, is a single-stranded glycoprotein having a Gla domain, an EGF domain and a protease domain in this order from the N-terminal end. The EGF domain of the factor IX is constituted by a sequence (SEQ ID NO: 25) consisting of amino acids at positions 90 to 123 in the amino acid sequence shown in SEQ ID NO: 23. The inventors of the present invention have focused on the fact that the amino acid sequence constituting the EGF domain of the factor IX contains a region consisting of specific 12 amino acid residues: C-K-D-D-I—S—S—Y-E-C—W—C (SEQ ID NO: 3), as in the case of EGF3 (EGF-like domain) in the Del-1 protein described above, and have found that a peptide comprising this region can increase the efficiency of gene transfer. Namely, examples of a peptide derived from a natural product and falling within the above peptide (a) or (b) include the full-length factor IX, as well as a peptide fragment consisting of the amino acid sequence shown in SEQ ID NO: 3 or various partial fragments comprising this fragment, and a partial fragment consisting of the EGF domain or various partial fragments comprising this EGF domain. It should be noted that the nucleotide sequence of DNA encoding the full-length factor IX is shown in SEQ ID NO: 22, while the nucleotide sequence of DNA encoding the EGF domain of the factor IX is shown in SEQ ID NO: 24.

The inducing agent of the present invention may comprise a derivative of the above peptide (a) or (b) together with or instead of the peptide. Such a derivative is intended to encompass all derivatives which can be prepared from the above peptide (a) or (b), and examples include derivatives whose constituent amino acids are partially replaced with unnatural amino acids, and/or derivatives whose constituent amino acids (mainly side chains thereof) are partially chemically modified.

The inducing agent of the present invention may comprise a salt of the above peptide (a) or (b) and/or a derivative of the peptide together with or instead of the peptide and/or the derivative. Such a salt is preferably a physiologically acceptable acid addition salt or basic salt. Examples of such an acid addition salt include salts with inorganic acids (e.g., hydrochloric acid, phosphoric acid, hydrobromic acid, sulfuric acid), as well as salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid). Examples of such a basic salt include salts with inorganic bases (e.g., sodium hydroxide, potassium hydroxide, ammonium hydroxide, magnesium hydroxide), as well as salts with organic bases (e.g., caffeine, piperidine, trimethylamine, pyridine).

The salt intended in the present invention can be prepared using a suitable acid such as hydrochloric acid or a suitable base such as sodium hydroxide. For example, the salt can be prepared by treatment in water or in a fluid containing an inert water-miscible organic solvent (e.g., methanol, ethanol or dioxane) according to a standard protocol.

Without being limited thereto, the inducing agent of the present invention may consist of the above peptide (a) or (b), a derivative thereof, or a salt of the peptide or the derivative, or may comprise the peptide, a derivative thereof, or a salt of the peptide or the derivative in combination with other components. Examples of other components include buffers such as PBS and Tris-HCl, as well as additives such as sodium azide and glycerol. In a case where the inducing agent of the present invention comprises other components, their content may be selected as appropriate within a range that does not significantly inhibit the ability to increase the efficiency of apoptosis induction provided by the above peptide, a derivative thereof, or a salt of the peptide or the derivative. More specifically, in the case of using the above peptide in a solution state, the peptide concentration is preferably, but not limited to, 0.3 ng/ml or higher, more preferably 0.3 to 5 ng/ml, even more preferably 0.3 to 2 ng/ml, still even more preferably 0.4 to 1.5 ng/ml, particularly preferably 0.6 to 1 ng/ml, and most preferably 0.8 to 1 ng/ml.

Target cells of apoptosis induced by the inducing agent of the present invention are not limited in any way and encompass various cells including tumor cells and other cells, with adherent cells being particularly preferred. Examples include Cos cells, CRL cells (human melanoma cell line), P5 cells (vascular endothelial cell line), KN cells (human squamous cell carcinoma cell line), etc. In the present invention, tumor cells are preferred for use as target cells of apoptosis induction, and specific examples of tumor cells preferably include CRL cells as mentioned above, as well as Cos cells, P5 cells, KN cells (human squamous cell carcinoma cell line), etc.

For induction of apoptosis in a target cell, it is not necessarily required to introduce the inducing agent of the present invention into the cell. When simply added from the outside of the cell, the inducing agent of the present invention can induce apoptosis in the cell. For this reason, apoptosis induction in various tumor cells can be achieved in a simple manner never before possible and in a wider range of cell types (cancer types). The inducing agent of the present invention is very useful in cancer treatment and/or studies thereof, by way of example.

The inducing agent of the present invention is characterized in that the above peptide (a) or (b) or the like can remain in the surrounding area (e.g., cancer lesion) of target cells where apoptosis is to be induced. Moreover, the above peptide (a) or (b) or the like is also characterized by having the ability to improve the efficiency of gene transfer into a cell. Thus, for example, if the inducing agent of the present invention is used for cancer treatment in combination with gene therapy, the inducing agent of the present invention (the above peptide (a) or (b) or the like) remaining in the lesion will exert an improving effect on the efficiency of gene transfer into residual diseased cells, e.g., when the diseased cells are required to repeatedly receive gene transfer. As a result, the inducing agent of the present invention allows gene therapy with high therapeutic effect.

The present invention can also provide a method for inducing apoptosis by using the inducing agent of the present invention. This method comprises the step of treating a target cell or a target cell population (tissue, organ) with the inducing agent of the present invention, and may further comprise any other non-limiting step(s). Treatment with the inducing agent of the present invention is intended to mean that the inducing agent of the present invention is contacted with a target cell or a target cell population (i.e., added to the cell or cell population and contacted with the outer surface thereof) or that the inducing agent of the present invention is introduced into a target cell. Above all, preferred is the former embodiment where the inducing agent is added. To introduce the inducing agent of the present invention into a target cell, the above peptide (a) or (b) serving as an active ingredient in the inducing agent of the present invention may be introduced in any way, either directly into the cell or in a state of DNA encoding the peptide (gene transfer). DNA introduction may be accomplished by using various known techniques for gene transfer, including liposome method (lipoplex method), polyplex method, peptide method, electroporation, and virus vector method.

### 2. DNA, Recombinant Vector, and Transformant

### (1) DNA

The present invention also contemplates DNA which comprises a nucleotide sequence encoding an amino acid sequence constituting the above peptide (a) or (b). Without being limited thereto, such DNA may consist only of a nucleotide sequence encoding the above peptide (a) or (b) or may comprise this nucleotide sequence and additional known nucleotide sequences required for gene expression (e.g., transcription promoter, SD sequence, Kozak sequence, terminator). In the nucleotide sequence encoding the above peptide (a) or (b), the type of codon is not limited in any way. For example, after transcription, it is possible to use codons generally used in mammals (e.g., humans) or codons generally used in microorganisms (e.g., E. coli or yeast), plants or the like, which may be selected or designed as appropriate.

Moreover, the present invention also contemplates DNA which is hybridizable under stringent conditions with DNA consisting of a nucleotide sequence complementary to DNA comprising a nucleotide sequence encoding the above peptide (a) or (b) and which encodes a protein having apoptosis-inducing activity. As used herein, the term “stringent conditions” refers to, for example, a salt (sodium) concentration of 150 to 900 mM and a temperature of 55° C. to 75° C., preferably a salt (sodium) concentration of 150 to 200 mM and a temperature of 60° C. to 70° C.

### (2) Recombinant Vector Comprising DNA

The present invention also contemplates a recombinant vector carrying the above DNA of the present invention ligated to (inserted into) an appropriate vector. The DNA of the present invention may be inserted into any vector capable of replicating in a host, and examples of such a vector include plasmid DNAs, phage DNAs, viruses, etc.

Examples of plasmid DNAs include E. coli-derived plasmids, Bacillus subtilis-derived plasmids, yeast-derived plasmids and so on, while examples of phage DNAs include λ phage and so on. Likewise, examples of viruses include adenoviruses and retroviruses, etc.

In addition to a promoter and the DNA of the present invention, the recombinant vector of the present invention may further comprise a cis element (e.g., enhancer), a splicing signal, a poly(A) addition signal, a ribosome binding sequence (SD sequence), a selection marker gene, a reporter gene and so on, if desired. It should be noted that examples of a selection marker gene include the dihydrofolate reductase gene, the ampicillin resistance gene, the neomycin resistance gene, etc. Examples of a reporter gene include genes for green fluorescent protein (GFP) or mutants thereof (fluorescent proteins such as EGFP, BFP and YFP), luciferase, alkaline phosphatase, LacZ, etc.

### (3) Transformant

The present invention also contemplates a transformant obtainable by introducing the above recombinant vector of the present invention into a host such that a desired gene can be expressed. Any host may be used for this purpose as long as it is capable of expressing the DNA of the present invention, and it is possible to use bacteria, yeast and other microorganisms well known in the art, by way of example.

In the case of using a bacterium as a host, the recombinant vector of the present invention is not only autonomously replicable in the bacterium, but may also comprise a promoter, a ribosome binding sequence, the DNA of the present invention, and a transcription termination sequence. Examples of bacteria include E. coli (Escherichia coli) and so on. Examples of a promoter available for use include the lac promoter and so on. Techniques used for vector introduction into bacteria include various known introduction techniques, such as calcium ion method.

In the case of using yeast as a host, Saccharomyces cerevisiae or the like may be used, by way of example. In this case, any promoter may be used as long as it allows expression in yeast, and examples of such a promoter include the gall promoter and so on. Techniques used for vector introduction into yeast include, for example, electroporation, spheroplast method and so on.

### 3. Pharmaceutical Composition

The inducing agent of the present invention (which can also be referred to as the peptide shown in (a) or (b) above) is useful as an active ingredient in pharmaceutical compositions.

Such pharmaceutical compositions are useful as those for cancer (tumor) treatment. In particular, because of having apoptosis-inducing activity in tumor cells or the like, the inducing agent of the present invention is preferred for use in tumor treatment. Namely, the inducing agent of the present invention is useful as an active ingredient in therapeutic agents for tumors.

Moreover, the inducing agent of the present invention can also be combined with existing antitumor agents (therapeutic agents for tumors) for use as active ingredients in pharmaceutical compositions for cancer treatment. In this case, the inducing agent of the present invention can eventually enhance the antitumor effect (antitumor activity) of the existing antitumor agents through its apoptosis-inducing activity. Thus, the present invention enables the provision of an enhancer comprising the inducing agent of the present invention for use in enhancing the antitumor effect of an antitumor agent, and a method for enhancing the antitumor effect of an antitumor agent by using the inducing agent of the present invention. Such an antitumor agent includes all known ones, as exemplified by, but not limited to, FasL (Fas ligand), TNFα, mitomycin and taxol, etc.

The pharmaceutical composition of the present invention is preferably provided in the form of a pharmaceutical composition comprising the inducing agent of the present invention as an active ingredient and further comprising a pharmaceutically acceptable carrier.

Specific examples of target cancer (tumor) to be treated by the pharmaceutical composition of the present invention include lung cancer (e.g., small cell lung cancer), large bowel cancer, breast cancer, liver cancer, kidney cancer, ovarian cancer, neuroendocrine tumor, neuroblastoma, glioma, neurofibromatosis type 1, gastric cancer, large bowel cancer, pancreatic cancer, bladder cancer, skin cancer, etc., with human cancers (tumors) of these types being preferred.

The term “pharmaceutically acceptable carrier” is intended to include excipients, diluents, extenders, disintegrants, stabilizers, preservatives, buffering agents, emulsifiers, aromatics, coloring agents, sweeteners, thickeners, correctives, solubilizers, as well as other additives, etc. One or more such carriers may be used to prepare pharmaceutical compositions in the form of injections, solutions, capsules, suspensions, emulsions or syrups, etc. These pharmaceutical compositions may be administered orally or parenterally. Other dosage forms for parenteral administration include, for example, injections which comprise one or more active agents and are formulated in a routine manner. In the case of injections, they may be prepared by being dissolved or suspended in a pharmaceutically acceptable carrier such as physiological saline or commercially available injectable distilled water. In a case where the inducing agent of the present invention (the peptide shown in (a) or (b) above) is administered in vivo, a colloidal dispersion system may also be used. Such a colloidal dispersion system can be expected to provide some effects, such as increased in vivo stability of the above peptide and/or efficient delivery of a compound to a specific organ, tissue or cell. The colloidal dispersion system is not limited in any way as long as it is commonly used, and examples include polyethylene glycol, polymer complexes, polymer assemblies, nanocapsules, microspheres, beads, as well as lipid-based dispersion systems including oil-in-water emulsions, micelles, mixed micelles and liposomes. Preferred are liposomes or artificial membrane vesicles which allow efficient delivery of a compound to a specific organ, tissue or cell.

The dose of the pharmaceutical composition of the present invention may vary depending on the age, sex, body weight and symptom of a patient, the intended therapeutic effect, the mode of administration, the time of treatment, or the type of the inducing agent of the present invention (the peptide shown in (a) or (b) above) to be contained in the pharmaceutical composition, etc. In general, the pharmaceutical composition of the present invention may be administered, but not limited to, at a single dose ranging from 100 μg to 5000 mg per adult.

For example, when administered in the form of injections, the pharmaceutical composition of the present invention may be administered at a single dose of 100 μg to 100 mg per kg body weight of a human patient on an average of one to several times a day. The mode of administration includes intravenous injection, subcutaneous injection, intracutaneous injection, intramuscular injection or intraperitoneal injection, with intravenous injection being preferred. In some cases, injections may be prepared as non-aqueous dilutions (e.g., polyethylene glycol, vegetable oils such as olive oil, alcohols such as ethanol), suspensions or emulsions. Such injections may be sterilized, for example, by being filtered through a filter or mixed with a disinfectant. Injections may be prepared in reconstitutable form. Namely, injections may be converted into sterile solid compositions by lyophilization or other techniques, and the resulting solid compositions may be dissolved in sterile injectable distilled water or other solvents before use.

It should be noted that the present invention also provides the use of the above inducing agent of the present invention (or the peptide shown in (a) or (b) above) for the manufacture of a medicament (drug) for cancer (tumor) treatment. Moreover, the present invention also provides the above inducing agent of the present invention (or the peptide shown in (a) or (b) above) for use in cancer (tumor) treatment.

Further, the present invention provides a method for cancer (tumor) treatment, characterized in that the above inducing agent of the present invention (or the peptide shown in (a) or (b) above) is used (i.e., administered to a patient), and also provides the use of the above inducing agent of the present invention (or the peptide shown in (a) or (b) above) for cancer (tumor) treatment.

### 4. Kit for Apoptosis Induction

The present invention also provides a kit for apoptosis induction, which comprises the inducing agent of the present invention as a constituent member. The kit of the present invention can be used for simple and effective induction of apoptosis in various cells including tumor cells and other cells, and is therefore significantly useful not only in the field of cancer treatment as described above, but also in the fields of various experiments, researches, etc.

### EXAMPLES

The present invention will be further described in more detail by way of the following examples, which are not intended to limit the scope of the present invention.

### Example 1

### Apoptosis Induction by Del-1 (1)

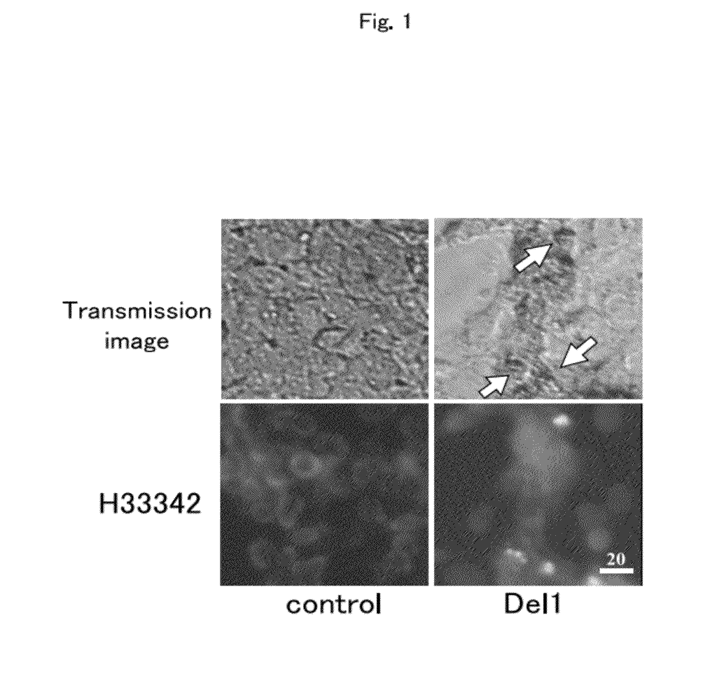

cDNA for full-length Del-1 (SEQ ID NO: 4) was integrated into an expression vector, pcDNA3 (Invitrogen), and then introduced into cultured Cos cells. As a control, pcDNA3 free from the cDNA for full-length Del-1 was used. At 24 hours after gene transfer, the cells were stained with H33342 (Hoechst) for cell nuclear staining and observed under a phase contrast microscope (transmission image) and a fluorescent microscope (H33342). The results obtained are shown in FIG. 1.

As indicated with arrows in the figure, in the cultured Cos cells receiving gene transfer, some cells were being released from the bottom surface of the culture vessel, as can be seen from their transmission image under the phase contrast microscope, and their nuclei were strongly stained with H33342, thus indicating that their chromosomes were aggregated.

### Example 2

### Apoptosis Induction by Del-1 (2)

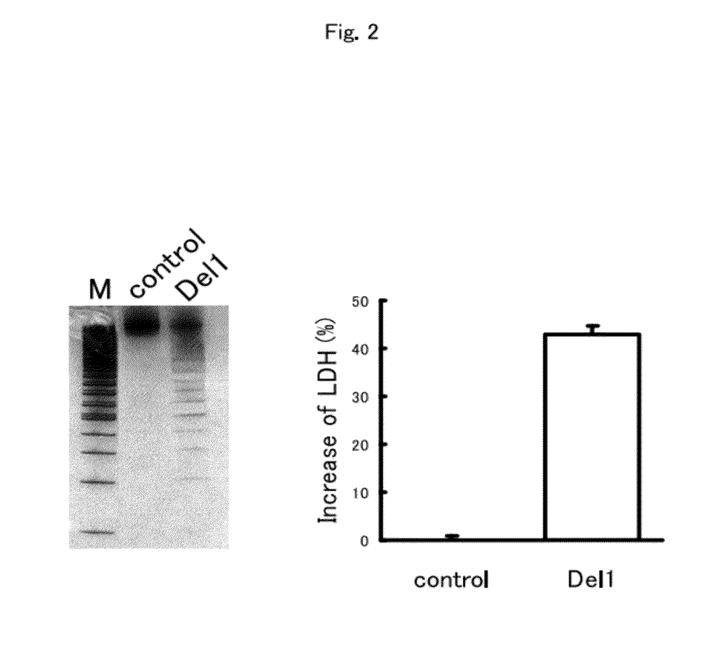

cDNA for full-length Del-1 (SEQ ID NO: 4) was integrated into an expression vector, pcDNA3 (Invitrogen), and then introduced into cultured Cos cells. As a control, pcDNA3 free from the cDNA for full-length Del-1 was used. For normalization of the gene transfer rate, DNA carrying the LacZ gene was also introduced simultaneously. The results obtained are shown in FIG. 2 (left and right panels).

Left panel: Chromosomal DNAs were taken from the cells and electrophoresed on a polyacrylamide gel, followed by silver staining. A ladder pattern indicative of apoptosis was observed in a sample of the cells forced to express Del-1.

Right panel: Culture supernatants were measured for lactate dehydrogenase (LDH) activity. LDH, which is an enzyme generally found in the cytoplasm, was found to leak into the culture supernatant upon cell death.

### Example 3

### Apoptosis Induction by Del-1 (3)

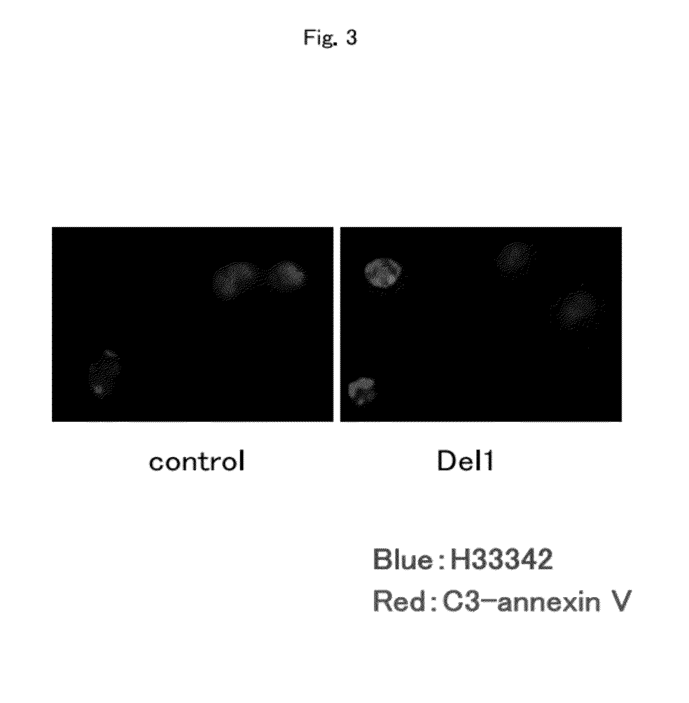

cDNA for full-length Del-1 (SEQ ID NO: 4) was integrated into an expression vector and then introduced into cultured Cos cells. At 24 hours after gene transfer, the cells were stained with H33342 and with C3-annexin V (annexin V conjugated with the fluorescent dye C3) and then observed under a fluorescent microscope. The results obtained are shown in FIG. 3.

The cell membrane of cells whose chromosomes were found to aggregate, as analyzed by H33342 staining, was stained with C3-annexin V, indicating that these cells underwent apoptosis. It should be noted that annexin V is capable of binding to phosphatidylserine, a component of cell membranes, while phosphatidylserine is known to leak (to be localized) outside of the cell membrane when the cells undergo apoptosis.

### Example 4

### Apoptosis Induction by Del-1 (4)

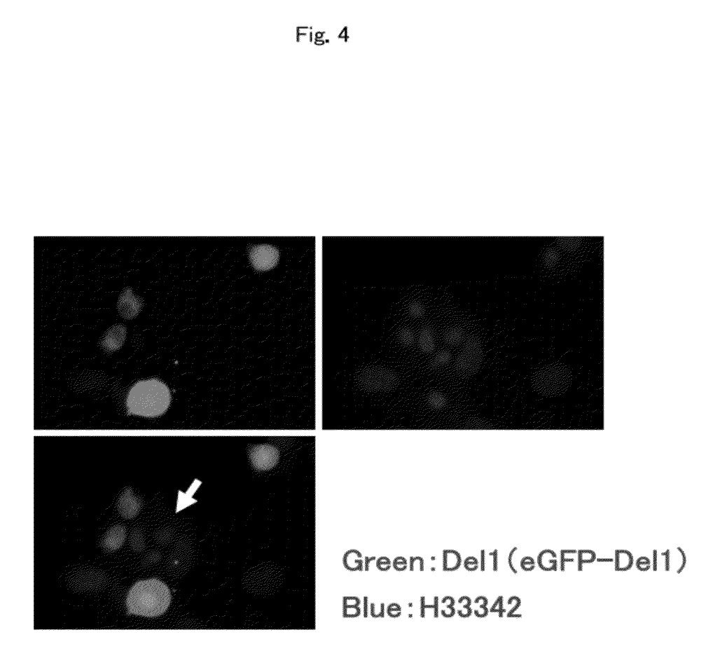

An expression vector designed to carry cDNA encoding eGFP-Del-1 (a fusion protein between eGFP and full-length Del-1) was introduced into cultured Cos cells. At 24 hours after gene transfer, the cells were fixed and stained with H33342. The results obtained are shown in FIG. 4.

The cells receiving Del-1 gene transfer (i.e., the cells expressing the above fusion protein) were observed in green by the action of eGFP. These cells were found to undergo apoptosis because they showed chromosomal aggregation. Although the three cells indicated with the arrow in the figure showed no green fluorescence and did not express Del-1 (i.e., not receive Del-1 gene transfer), they showed chromosomal aggregation. This would mean that Del-1 secreted from the cells receiving Del-1 gene transfer would also act on their adjacent cells to thereby induce apoptosis in these cells.

### Example 5

### Apoptosis Induction by Del-1 (5)

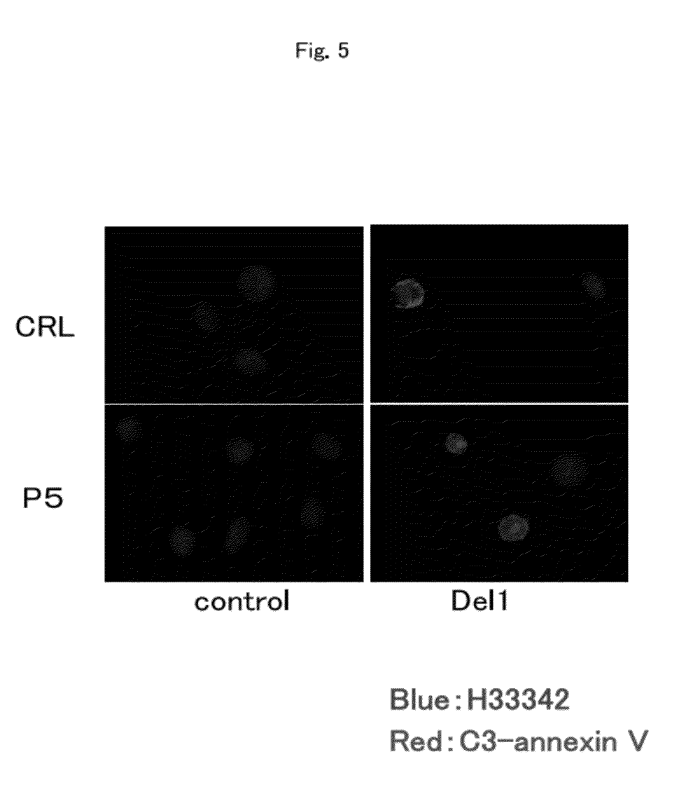

cDNA for full-length Del-1 (SEQ ID NO: 4) was integrated into an alkaline phosphatase (AP) expression vector (APtag4) and then introduced into cultured Cos cells. As a control, an AP expression vector free from the cDNA for full-length Del-1 was used. The resulting culture supernatant was measured for AP activity, normalized for AP fusion protein concentration and then added to a culture of CRL cells (human melanoma cells) or P5 cells (vascular endothelial cell line) (instead of using direct gene transfer, Cos cells were allowed to produce a recombinant protein and the resulting conditioned medium was used, because CRL and P5 were low in the efficiency of gene transfer). At 24 hours after addition, the cells were stained with H33342 and with C3-annexin V for observation. The results obtained are shown in FIG. 5.

The cell membrane of cells whose chromosomes were found to aggregate, as analyzed by H33342 staining, was stained with C3-annexin V, indicating that these cells underwent apoptosis.

### Example 6

### Apoptosis Induction by Del-1 (6)

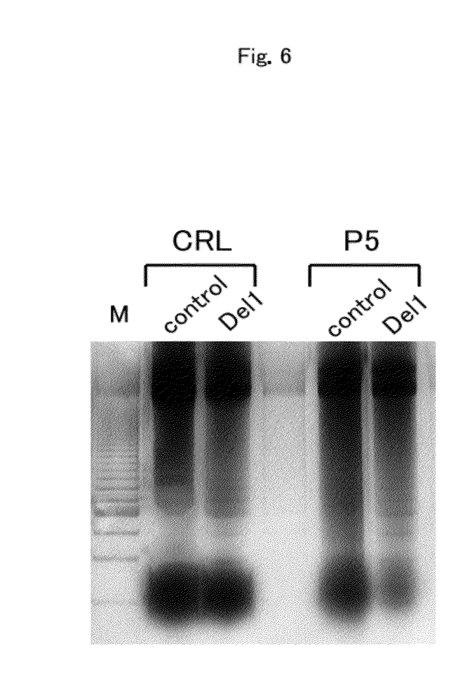

cDNA for full-length Del-1 (SEQ ID NO: 4) was integrated into an AP expression vector (APtag4) and then introduced into cultured Cos cells. As a control, an AP expression vector free from the cDNA for full-length Del-1 was used. The resulting culture supernatant was measured for AP activity, normalized for AP fusion protein concentration and then added to a culture of CRL cells (human melanoma cells) or P5 cells (vascular endothelial cell line) (instead of using direct gene transfer, Cos cells were allowed to produce a recombinant protein and the resulting conditioned medium was used, because CRL and P5 were low in the efficiency of gene transfer). At 24 hours after addition, chromosomal DNAs were taken from the cells and electrophoresed on a polyacrylamide gel, followed by silver staining. The results obtained are shown in FIG. 6.

A ladder pattern indicative of apoptosis was observed in a sample of the cells forced to express Del-1.

### Example 7

### Active Center in Apoptosis Induction (1)

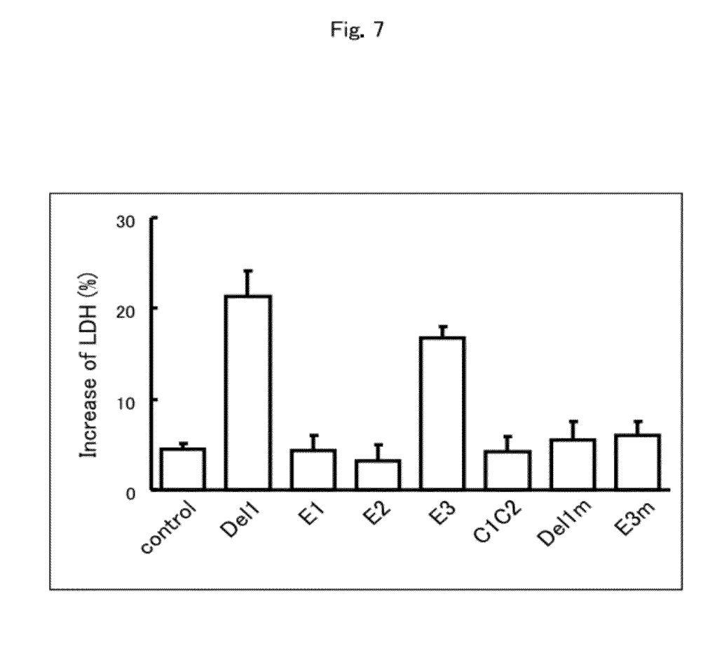

cDNA for full-length Del-1 (SEQ ID NO: 4) was integrated into an AP expression vector (APtag4) and then introduced into cultured Cos cells. Likewise, cDNA for a part of Del-1 was also integrated into APtag4 and then introduced into cultured Cos cells. As a control, an AP expression vector free from the above cDNA for full-length Del-1 or a part thereof was used. It should be noted that the members each used as a part of Del-1 were EGF-like domains in the full-length Del-1 protein, i.e., EGF1 (E1; cDNA: SEQ ID NO: 26, amino acid sequence: SEQ ID NO: 27), EGF2 (E2; cDNA: SEQ ID NO: 28, amino acid sequence: SEQ ID NO: 29) and EGF3 (E3; cDNA: SEQ ID NO: 6, amino acid sequence: SEQ ID NO: 7), a fusion protein between discoidin I-like domains, i.e., Discoidin1 and Discoidin2 (C1C2; cDNA: SEQ ID NO: 30, amino acid sequence: SEQ ID NO: 31), an amino acid substitution (D136E) mutant of the above full-length Del-1 (Del1m; cDNA: SEQ ID NO: 32, amino acid sequence: SEQ ID NO: 33), as well as an amino acid substitution (D136E) mutant of E3, i.e., E3 D136E (E3m; cDNA: SEQ ID NO: 34, amino acid sequence: SEQ ID NO: 35). It should be noted that the expression “136” in “D136E” which represents an embodiment of amino acid substitution in E3m denotes the position of the 136th amino acid residue in the amino acid sequence of full-length Del-1 (SEQ ID NO: 5), which corresponds to the position of the 14th amino acid residue in the amino acid sequence of E3 (SEQ ID NO: 7). For normalization of the gene transfer rate, DNA carrying the LacZ gene was also introduced simultaneously. At 24 hours after gene transfer, the medium in each case was replaced with fresh culture medium and, after further one hour, the supernatant was measured for LDH activity. A cell lysate was prepared for each case and measured for LacZ activity. Assuming that the LacZ activity of the cells transformed with cDNA for full-length Del-1 was set to 1, increases in LDH activity were normalized. The results obtained are shown in FIG. 7.

The supernatant of the cells transformed with cDNA for E3 was found to show an increase in LDH activity similar to that of the cells transformed with cDNA for full-length Del-1. This effect was lost by D136E mutation, as can be seen from the results obtained for the cells transformed with cDNA for E3m.

### Example 8

### Active Center in Apoptosis Induction (2)

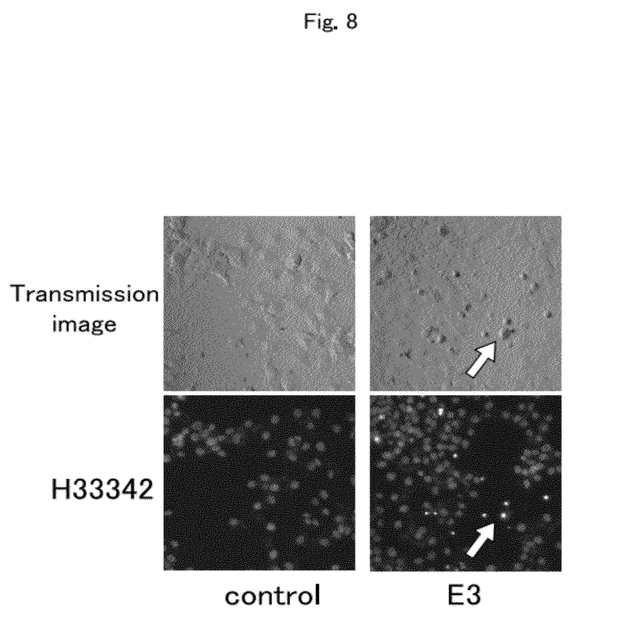

To cultured Cos cells, a recombinant E3 protein produced from transformed E. coli was added. At 24 hours after addition, the cells were stained with H33342 and observed under a phase contrast microscope and a fluorescent microscope. The results obtained are shown in FIG. 8.

As indicated with arrows in the figure, in the cultured Cos cells after addition of the recombinant protein, some cells were being released from the bottom surface of the culture vessel, as can be seen from their transmission image under the phase contrast microscope, and their nuclei were strongly stained with H33342, thus indicating that their chromosomes were aggregated.

### Example 9

### Consensus Sequence

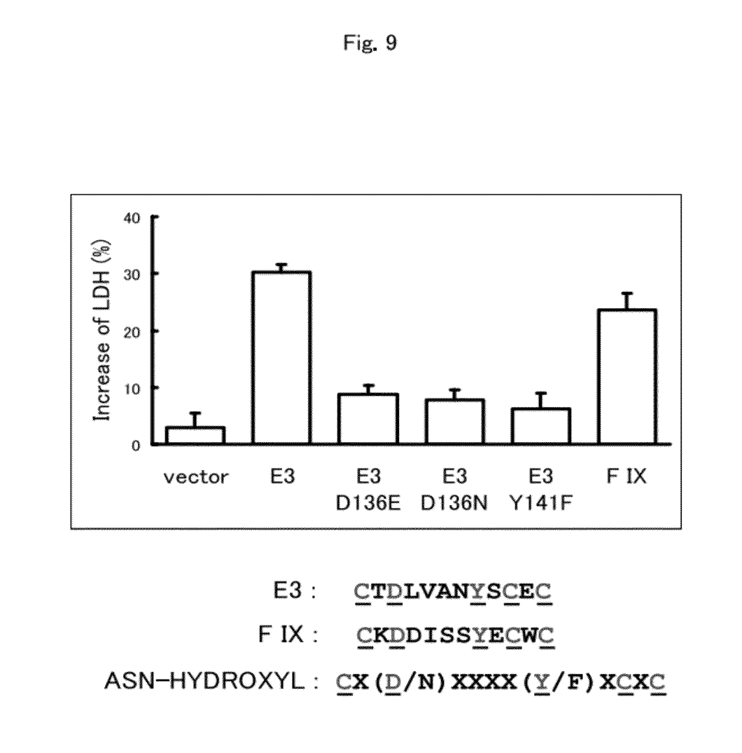

cDNA for E3 (a part of Del-1) or an amino acid substitution mutant thereof was integrated into an AP expression vector (APtag4) and then introduced into cultured Cos cells. Likewise, cDNA for the EGF domain of blood coagulation factor IX (F IX; cDNA: SEQ ID NO: 24, amino acid sequence: SEQ ID NO: 25) was also integrated into APtag4 and then introduced into cultured Cos cells. As a control, an AP expression vector free from the above cDNAs for E3 and so on was used. It should be noted that the amino acid substitution mutants of E3 used for the above purpose were E3 D136E (cDNA: SEQ ID NO: 34, amino acid sequence: SEQ ID NO: 35), E3 D136N (cDNA: SEQ ID NO: 36, amino acid sequence: SEQ ID NO: 37) and E3 Y141F (cDNA: SEQ ID NO: 38, amino acid sequence: SEQ ID NO: 39). It should be noted that the expressions “136” and “141” in “D136E”, “D136N” and “Y141F” which represent embodiments of amino acid substitution in the E3 mutants denote the positions of the 136th and 141st amino acid residues, respectively, in the amino acid sequence of full-length Del-1 (SEQ ID NO: 5), which correspond to the positions of the 14th and 19th amino acid residues, respectively, in the amino acid sequence of E3 (SEQ ID NO: 7). For normalization of the gene transfer rate, DNA carrying the LacZ gene was also introduced simultaneously. At 4 hours after gene transfer, the medium in each case was replaced with fresh culture medium and, after further 24 hours, the supernatant was measured for LDH activity. A cell lysate was prepared for each case and measured for LacZ activity. Assuming that the LacZ activity of the cells transformed with E3 (wild-type) was set to 1, increases in LDH activity were normalized. The results obtained are shown in FIG. 9.

The LDH activity was reduced in all cases transformed with the E3 mutants. This result indicated that the LDH activity was reduced by mutations in amino acid residues (D and Y in this example) corresponding to the specific amino acid residues (C, D, Y, C and C (amino acid residues at positions 1, 3, 8, 10 and 12 in SEQ ID NO: 40)) in the amino acid sequence (C—X-[D/N]—X—X—X—X—[Y/F]—X—C—X—C (SEQ ID NO: 40)) constituting the sequence for hydroxylation of asparagine residues (ASN-HYDROXYL). In contrast, the cells transformed with F IX (which has no amino acid residue in common with E3, except for the above specific amino acid residues) were found to have high LDH activity, as in the case of the E3-transformed cells.

### Example 10

### Concentration Dependence

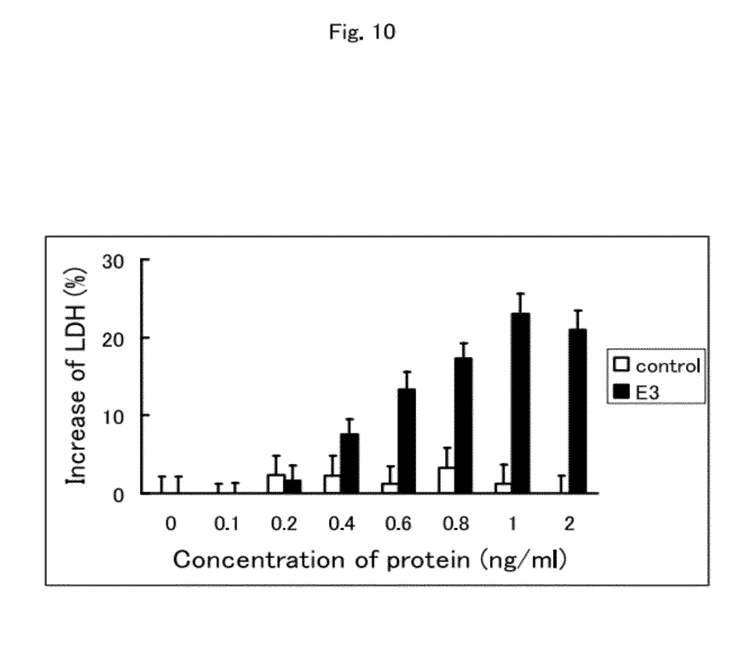

A recombinant E3 protein produced from transformed E. coli was added at different concentrations (0 to 2.0 ng/ml) to cultured Cos cells. At 24 hours after addition, the culture supernatants were each measured for LDH activity. The results obtained are shown in FIG. 10.

E3 protein concentrations of 0.4 ng/ml or higher were found to be effective for apoptosis induction. It should be noted that the effect on apoptosis induction remained unchanged at E3 protein concentrations higher than 1 ng/ml.

### Example 11

### Effectiveness of E3 in Various Cells

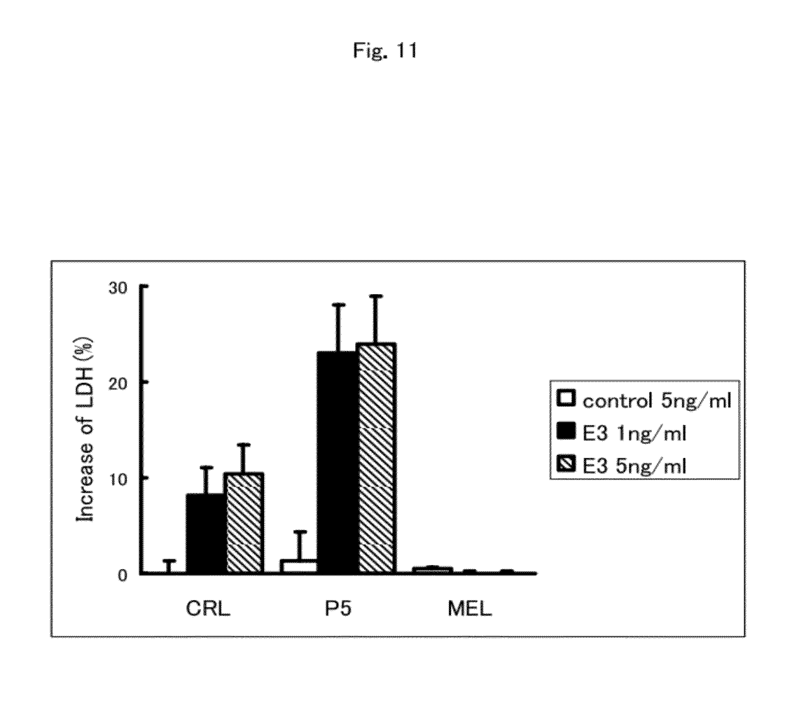

A recombinant E3 protein produced from transformed E. coli was added at a concentration of 1 ng/ml or 5 ng/ml to cultured Cos cells. At 24 hours after addition, the culture supernatants were each measured for LDH activity. The results obtained are shown in FIG. 11.

The E3 protein showed cytotoxic activity (apoptosis induction) against CRL cells (human melanoma cells) and P5 cells (vascular endothelial cell line), which are adherent cells, whereas the E3 protein showed no cytotoxic activity against MEL cells (mouse leukemia cells), which are floating cells.

### Example 12

### Therapeutic Effect on Tumor Provided by EGF3 Domain-Containing Peptide

Using tumor-transplanted model mice, a peptide containing the EGF3 domain, which is a part of Del-1, was studied for its therapeutic effect on tumor. More specifically, this peptide was verified as to whether its combined use with the FasL (Fas ligand) protein, whose antitumor effect has already been established, would enhance the antitumor effect when compared to FasL alone.

First, cDNAs shown in (A) and (B) below were each integrated into an expression vector, pcDNA3D (Invitrogen), and then cloned to prepare two recombinant DNAs.
(A) cDNA for full-length mouse FasL (cDNA (GenBank accession No. NM_{—}010177): SEQ ID NO: 43, amino acid sequence (GenBank accession No. NP_{—}034307): SEQ ID NO: 44)
(B) cDNA in (A) above and cDNA for a fusion peptide between EGF3 domain and Discoidin1 (E3C1; cDNA: SEQ ID NO: 41, amino acid sequence: SEQ ID NO: 42) (two cDNAs in total)

It should be noted that E3C1 is a peptide consisting of amino acids at positions 123 to 319 of the amino acid sequence for full-length mouse Del-1 (SEQ ID NO: 5) (i.e., a peptide encoded by the nucleotide sequence located at positions 985 to 1575 of the nucleotide sequence for full-length mouse Del-1 (SEQ ID NO: 4)).

Then, 5×10⁶ SCCKN cells (human oral squamous cell carcinoma-derived cell line) were injected under the dorsal skin of nude mice at 5 weeks of age. The mice were then measured over time for their tumor size. At the time when the tumor size exceeded 10 mm×10 mm, the mice were intratumorally injected (gene transfer) with the two recombinant DNAs prepared above to thereby express each cDNA in the tumor cells. During this gene transfer, in vivo jet-PEI (Polyplus Transfection) was used as a gene transfer reagent and the amount of DNA to be introduced was set to 10 μg. Four mice were used for introduction of the recombinant DNA containing the cDNA shown in (A) above, while five mice were used for introduction of the recombinant DNA containing the cDNAs shown in (B) above.

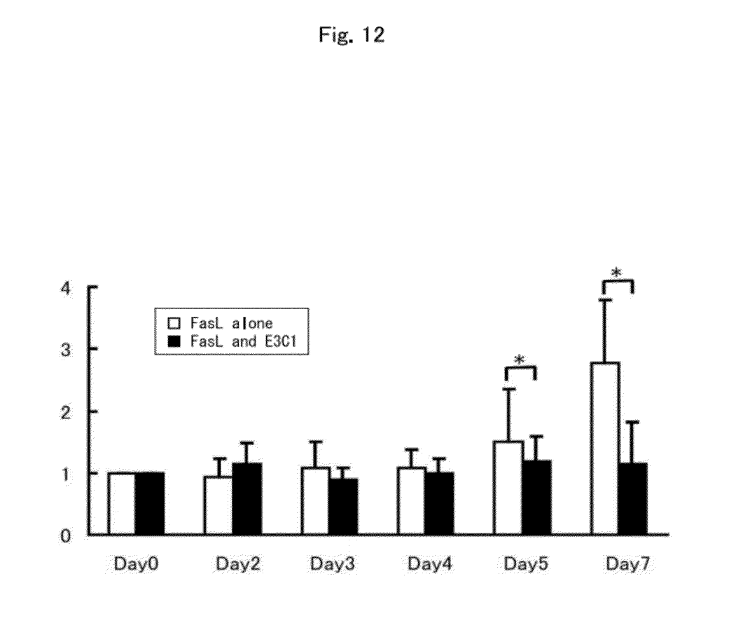

The tumor size after gene transfer was measured over time (for 7 days: Day0 to Day7), and the results obtained are shown in FIG. 12. Introduction together with the cDNA for E3C1 provided a higher antitumor effect, thus indicating that E3C1 had an enhancing effect on the antitumor effect of FasL.

### INDUSTRIAL APPLICABILITY

The present invention enables the provision of apoptosis-inducing agents which induce apoptosis in various cells including tumor cells and other cells, etc.

The apoptosis-inducing agents of the present invention can induce apoptosis in a wide range of cell types in a simple manner, and also effectively induce apoptosis in tumor cells. Thus, the apoptosis-inducing agents of the present invention are very useful in that they can be effectively used, e.g., for pharmaceutical compositions or methods for cancer treatment.

Moreover, the apoptosis-inducing agents of the present invention are also advantageous in that they remain in the surrounding area (e.g., lesion) of target cells and are capable of improving the efficiency of gene transfer in the subsequent gene therapy, etc. Thus, the apoptosis-inducing agents of the present invention are very useful in that they can provide, for example, pharmaceutical compositions or methods for cancer treatment with high therapeutic effect when used in combination with agents for gene therapy.

### Sequence Listing Free Text

SEQ ID NO: 1: Peptide

SEQ ID NO: 1: Xaa at locations 2, 4, 5, 6, 7, 9 and 11 represents any amino acid residue.

SEQ ID NO: 40: Peptide

SEQ ID NO: 40: Xaa at locations 2, 4, 5, 6, 7, 9 and 11 represents any amino acid residue.

SEQ ID NO: 40: Xaa at location 3 represents aspartic acid or asparagine.

SEQ ID NO: 40: Xaa at location 8 represents tyrosine or phenylalanine.

1. An apoptosis-inducing agent, which comprises a peptide shown in (a) or (b) below, a derivative thereof, or a salt of the peptide or the derivative:
(a) a peptide which comprises an amino acid sequence represented by the following formula (I):
(I)
(SEQ ID NO: 1)
C-X-D-X-X-X-X-Y-X-C-X-C
(wherein X represents any amino acid residue, C represents cysteine, D represents aspartic acid, and Y represents tyrosine); or
(b) a peptide which comprises an amino acid sequence with deletion, substitution or addition of one or several amino acids in the amino acid sequence represented by formula (I) and which has apoptosis-inducing activity.

2. The inducing agent according to claim 1, wherein the amino acid sequence represented by formula (I) is the amino acid sequence shown in SEQ ID NO: 2 or SEQ ID NO: 3.

3. The inducing agent according to claim 1, wherein the peptide shown in (a) is a peptide comprising the amino acid sequence shown in SEQ ID NO: 5 or SEQ ID NO: 7.

4. A method for inducing apoptosis, which comprises the step of treating a target cell or a target cell population with the inducing agent according to claim 1.

5. The method according to claim 4, wherein the step is intended to contact the target cell or target cell population with the inducing agent.

6. A pharmaceutical composition for cancer treatment, which comprises the inducing agent according to claim 1.

7. An apoptosis-inducing agent, which comprises a peptide consisting of 12 to 100 amino acid residues comprising the amino acid sequence shown in SEQ ID NO: 2, a derivative thereof, or a salt of the peptide or the derivative.

8. An apoptosis-inducing agent, which comprises a peptide consisting of the amino acid sequence shown in SEQ ID NO: 2, a derivative thereof, or a salt of the peptide or the derivative.

9. An apoptosis-inducing agent, which comprises a peptide consisting of the amino acid sequence shown in SEQ ID NO: 7, a derivative thereof, or a salt of the peptide or the derivative.

10. An apoptosis-inducing agent, which comprises a peptide consisting of the amino acid sequence shown in SEQ ID NO: 19, a derivative thereof, or a salt of the peptide or the derivative.

11. An apoptosis-inducing agent, which comprises a peptide consisting of the amino acid sequence shown in SEQ ID NO: 17, a derivative thereof, or a salt of the peptide or the derivative.

12. An apoptosis-inducing agent, which comprises a peptide consisting of the amino acid sequence shown in SEQ ID NO: 21, a derivative thereof, or a salt of the peptide or the derivative.

13. An apoptosis-inducing agent, which comprises a peptide consisting of the amino acid sequence shown in SEQ ID NO: 42, a derivative thereof, or a salt of the peptide or the derivative.

14. A pharmaceutical composition for cancer treatment, which comprises the inducing agent according to claim 7.

15. An apoptosis-inducing agent, which comprises a peptide comprising the amino acid sequence shown in SEQ ID NO: 3, a derivative thereof, or a salt of the peptide or the derivative.

16. An apoptosis-inducing agent, which comprises a peptide consisting of the amino acid sequence shown in SEQ ID NO: 3, a derivative thereof, or a salt of the peptide or the derivative.

17. An apoptosis-inducing agent, which comprises a peptide consisting of the amino acid sequence shown in SEQ ID NO: 25, a derivative thereof, or a salt of the peptide or the derivative.

18. An apoptosis-inducing agent, which comprises a peptide consisting of the amino acid sequence shown in SEQ ID NO: 23, a derivative thereof, or a salt of the peptide or the derivative.

19. A pharmaceutical composition for cancer treatment, which comprises the inducing agent according to claim 16.


SEQUENCE LISTING (44 sequences)

SEQ ID NO: 1 - protein, 12 aa, Artificial Sequence
  Synthetic peptide
CXDXXXXYXCXC

SEQ ID NO: 2 - protein, 12 aa, Mus musculus
CTDLVANYSCEC

SEQ ID NO: 3 - protein, 12 aa, Mus musculus
CTDLVANYSCEC

SEQ ID NO: 4 - DNA, 2303 bp, Mus musculus
  CDS (619)..(2061)
gaattccggttaactgaggacaaagggtaatgcagaagtgatatttgatttccattctcattcccagtggccttgatatttaaactgattcctgccaccaggtccttgggccaccctgtccctgcgtctcatatttctgcatgctgctttgtttgtatatagtgcgctcctggcctcaggctcgctcccctccagctctcgcttcattgttctccaagtcagaagcccccgcatccgccgcgcagcagcgtgagccgtagtcactgctggccgcttcgcctgcgtgcgcgcacggaaatcggggagccaggaacccaaggagccgccgtccgcccgctgtgcctctgctagaccactcgcagccccagcctctctcaagcgcacccacctccgcgcaccccagctcaggcgaagctggagtgagggtgaatcaccctttctctagggccaccactcttttatcgcccttcccaagatttgagaagcgctgcgggaggaaagacgtcctcttgatctctgacagggcggggtttactgctgtcctgcaggcgcgcctcgcctactgtgccctccgctacgaccccggaccagcccaggtcacgtccgtgagaagggatcatgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaatgcctggacttccggccataacgaccagtcacaatggttacaggttgatcttcttgtccctactaaggtgacaggcatcattacacaaggagctaaagattttggtcacgtgcagtttgttgggtcatacaaactagcttacagcaatgatggagaacactggatggtgcaccaggatgaaaaacagaggaaagacaaggtttttcaaggcaattttgacaatgacactcacaggaaaaatgtcatcgaccctcccatctatgcacgattcataagaatccttccttggtcctggtatggaaggatcactctgcggtcagagctgctgggctgcgcagaggaggaatgaagtgcggggccgcacatcccacaatgcttttctttattttcctataagtatctccacgaaatgaactgtgtgaagctgatggaaactgcatttgtttttttcaaagtgttcaaattatggtaggctactgactgtctttttaggagttctaagcttgcctttttaataatttaatttggtttcctttgctcaactctcttatgtaatatcacactgtctgtgagttactcttcttgttctct

SEQ ID NO: 5 - protein, 480 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVNAWTSGHNDQSQWLQVDLLVPTKVTGIITQGAKDFGHVQFVGSYKLAYSNDGEHWMVHQDEKQRKDKVFQGNFDNDTHRKNVIDPPIYARFIRILPWSWYGRITLRSELLGCAEEE

SEQ ID NO: 6 - DNA, 105 bp, Mus musculus
  CDS (1)..(105)
tgtgaagctgagccttgcagaaatggcggaatatgtaccgaccttgttgctaactactcttgtgaatgcccaggagaatttatgggacgaaattgtcaatataaa

SEQ ID NO: 7 - protein, 35 aa, Mus musculus
CEAEPCRNGGICTDLVANYSCECPGEFMGRNCQYK

SEQ ID NO: 8 - DNA, 1044 bp, Mus musculus
  CDS (1)..(1044)
atgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatg

SEQ ID NO: 9 - protein, 348 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDM

SEQ ID NO: 10 - DNA, 1095 bp, Mus musculus
  CDS (1)..(1095)
atgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaat

SEQ ID NO: 11 - protein, 365 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVN

SEQ ID NO: 12 - DNA, 1104 bp, Mus musculus
  CDS (1)..(1104)
atgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaatgcctggact

SEQ ID NO: 13 - protein, 368 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVNAWT

SEQ ID NO: 14 - DNA, 1155 bp, Mus musculus
  CDS (1)..(1155)
atgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaatgcctggacttccggccataacgaccagtcacaatggttacaggttgatcttcttgtccct

SEQ ID NO: 15 - protein, 385 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVNAWTSGHNDQSQWLQVDLLVP

SEQ ID NO: 16 - DNA, 678 bp, Mus musculus
  CDS (1)..(678)
tgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatg

SEQ ID NO: 17 - protein, 226 aa, Mus musculus
CEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDM

SEQ ID NO: 18 - DNA, 285 bp, Mus musculus
  CDS (1)..(285)
tgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcag

SEQ ID NO: 19 - protein, 95 aa, Mus musculus
CEAEPCRNGGICTDLVANYSCECPGEFMGRNCQYKCSGPLGIEGGIISNQQITASSTHRALFGLRKWYPYYARLNKKGLINAWTAAENDRWPWIQ

SEQ ID NO: 20 - DNA, 678 bp, Homo sapiens
  CDS (1)..(678)
tgcgaagttgagccttgcaaaaatggtggaatatgtacagatcttgttgctaactattcctgtgagtgcccaggcgaatttatgggaagaaattgtcaatacaaatgctcaggcccactgggaattgaaggtggaattatatcaaaccagcaaatcacagcttcctctactcaccgagctctttttggactccaaaaatggtatccctactatgcacgtcttaataagaaggggcttataaatgcgtggacagctgcagaaaatgacagatggccgtggattcagataaatttgcaaaggaaaatgagagttactggtgtgattacccaaggagccaagaggattggaagcccagagtatataaaatcctacaaaattgcctacagtaatgatggaaagacttgggcaatgtacaaagtgaaaggcaccaatgaagacatggtgtttcgtggaaacattgataacaacactccatatgctaactctttcacaccccccataaaagctcagtatgtaagactctatccccaagtttgtcgaagacattgcactttgcgaatggaacttcttggctgtgaactgtcgggttgttctgagcctctgggtatgaaatcaggacatatacaagactatcagatcactgcctccagcatcttcagaacgctcaacatggacatg

SEQ ID NO: 21 - protein, 226 aa, Homo sapiens
CEVEPCKNGGICTDLVANYSCECPGEFMGRNCQYKCSGPLGIEGGIISNQQITASSTHRALFGLQKWYPYYARLNKKGLINAWTAAENDRWPWIQINLQRKMRVTGVITQGAKRIGSPEYIKSYKIAYSNDGKTWAMYKVKGTNEDMVFRGNIDNNTPYANSFTPPIKAQYVRLYPQVCRRHCTLRMELLGCELSGCSEPLGMKSGHIQDYQITASSIFRTLNMDM

SEQ ID NO: 22 - DNA, 1395 bp, Mus musculus
  CDS (1)..(1395)
atggcagaatccccggctctcatcaccatcttccttttaggatatctactcagtaccgaatgtgcagttttccttgatcgtgaaaatgccaccaaaattcttacccgtccaaagagatataattcaggaaaactagaagagtttgttcgaggaaaccttgaaagagagtgtatagaagaaagatgtagttttgaagaagcacgagaagtttttgaaaacactgaaaaaactactgaattttggaagcagtatgttgatggagatcagtgtgaatcaaatccttgtttaaatggtggaatatgcaaggatgatattagttcctatgaatgctggtgccaagttggatttgaaggaaggaactgtgaattagatgcaacgtgtaacattaaaaatggcaggtgcaagcagttttgtaaaaacagtcctgataacaaggtaatttgttcctgcactgagggataccaacttgcagaagaccagaagtcctgtgaaccaacagttccatttccatgtgggagagcttctatttcatacagttctaaaaagatcacgagagctgagactgttttctctaatatggactatgaaaattctactgaagctgtattcattcaagatgacatcactgatggtgccattcttaataacgtcactgaaagtagtgaatcacttaatgacttcactcgagttgttggtggagaaaacgcaaaaccgggtcaaatcccttggcaggtcattttaaatggtgaaattgaggcattctgtggaggtgccatcattaatgaaaaatggattgtaactgctgcccactgtcttaaacctggtgataaaattgaggttgttgctggtgaatataacattgataagaaggaagacacagaacaaaggagaaatgtgattcgaactatccctcatcaccagtacaatgcaactattaataagtatagtcatgacattgccttgctggaactggataaacctttaatactaaacagctatgtaacacctatctgtgttgccaatagggaatatacaaatatcttcctcaagtttggttctggctatgtcagtggctggggaaaagtcttcaacaaagggagacaggcttccattcttcagtaccttagagttccactggtggatagagccacatgccttaggtccacaacattcactatctataacaacatgttctgtgcaggctaccgtgaaggaggcaaagattcgtgtgaaggagatagtgggggaccccatgttactgaagtagaagggacaagtttcttaactggcattattagctggggtgaagaatgtgcaatgaaaggcaaatatggaatatatactaaggtttcccggtacgtcaactggattaaggaaaaaacaaagctaacttaa

SEQ ID NO: 23 - protein, 464 aa, Mus musculus
MAESPALITIFLLGYLLSTECAVFLDRENATKILTRPKRYNSGKLEEFVRGNLERECIEERCSFEEAREVFENTEKTTEFWKQYVDGDQCESNPCLNGGICKDDISSYECWCQVGFEGRNCELDATCNIKNGRCKQFCKNSPDNKVICSCTEGYQLAEDQKSCEPTVPFPCGRASISYSSKKITRAETVFSNMDYENSTEAVFIQDDITDGAILNNVTESSESLNDFTRVVGGENAKPGQIPWQVILNGEIEAFCGGAIINEKWIVTAAHCLKPGDKIEVVAGEYNIDKKEDTEQRRNVIRTIPHHQYNATINKYSHDIALLELDKPLILNSYVTPICVANREYTNIFLKFGSGYVSGWGKVFNKGRQASILQYLRVPLVDRATCLRSTTFTIYNNMFCAGYREGGKDSCEGDSGGPHVTEVEGTSFLTGIISWGEECAMKGKYGIYTKVSRYVNWIKEKTKLT

SEQ ID NO: 24 - DNA, 102 bp, Mus musculus
  CDS (1)..(102)
tgtgaatcaaatccttgtttaaatggtggaatatgcaaggatgatattagttcctatgaatgctggtgccaagttggatttgaaggaaggaactgtgaatta

SEQ ID NO: 25 - protein, 34 aa, Mus musculus
CESNPCLNGGICKDDISSYECWCQVGFEGRNCEL

SEQ ID NO: 26 - DNA, 156 bp, Mus musculus
  CDS (1)..(156)
tgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccc

SEQ ID NO: 27 - protein, 52 aa, Mus musculus
CNPNPCENGGICLSGLADDSFSCECPEGFAGPNCSSVVEVASDEEKPTSAGP

SEQ ID NO: 28 - DNA, 135 bp, Mus musculus
  CDS (1)..(135)
tgcatccctaacccatgccataacggaggaacctgtgagataagcgaagcctatcgaggagacacattcataggctatgtttgtaaatgtcctcggggatttaatgggattcactgtcagcacaatataaatgaa

SEQ ID NO: 29 - protein, 45 aa, Mus musculus
CIPNPCHNGGTCEISEAYRGDTFIGYVCKCPRGFNGIHCQHNINE

SEQ ID NO: 30 - DNA, 972 bp, Mus musculus
  CDS (1)..(972)
tgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaatgcctggacttccggccataacgaccagtcacaatggttacaggttgatcttcttgtccctactaaggtgacaggcatcattacacaaggagctaaagattttggtcacgtgcagtttgttgggtcatacaaactagcttacagcaatgatggagaacactggatggtgcaccaggatgaaaaacagaggaaagacaaggtttttcaaggcaattttgacaatgacactcacaggaaaaatgtcatcgaccctcccatctatgcacgattcataagaatccttccttggtcctggtatggaaggatcactctgcggtcagagctgctgggctgcgcagaggaggaatga

SEQ ID NO: 31 - protein, 323 aa, Mus musculus
CSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVNAWTSGHNDQSQWLQVDLLVPTKVTGIITQGAKDFGHVQFVGSYKLAYSNDGEHWMVHQDEKQRKDKVFQGNFDNDTHRKNVIDPPIYARFIRILPWSWYGRITLRSELLGCAEEE

SEQ ID NO: 32 - DNA, 2303 bp, Mus musculus
  CDS (619)..(2061)
gaattccggttaactgaggacaaagggtaatgcagaagtgatatttgatttccattctcattcccagtggccttgatatttaaactgattcctgccaccaggtccttgggccaccctgtccctgcgtctcatatttctgcatgctgctttgtttgtatatagtgcgctcctggcctcaggctcgctcccctccagctctcgcttcattgttctccaagtcagaagcccccgcatccgccgcgcagcagcgtgagccgtagtcactgctggccgcttcgcctgcgtgcgcgcacggaaatcggggagccaggaacccaaggagccgccgtccgcccgctgtgcctctgctagaccactcgcagccccagcctctctcaagcgcacccacctccgcgcaccccagctcaggcgaagctggagtgagggtgaatcaccctttctctagggccaccactcttttatcgcccttcccaagatttgagaagcgctgcgggaggaaagacgtcctcttgatctctgacagggcggggtttactgctgtcctgcaggcgcgcctcgcctactgtgccctccgctacgaccccggaccagcccaggtcacgtccgtgagaagggatcatgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaacttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaatgcctggacttccggccataacgaccagtcacaatggttacaggttgatcttcttgtccctactaaggtgacaggcatcattacacaaggagctaaagattttggtcacgtgcagtttgttgggtcatacaaactagcttacagcaatgatggagaacactggatggtgcaccaggatgaaaaacagaggaaagacaaggtttttcaaggcaattttgacaatgacactcacaggaaaaatgtcatcgaccctcccatctatgcacgattcataagaatccttccttggtcctggtatggaaggatcactctgcggtcagagctgctgggctgcgcagaggaggaatgaagtgcggggccgcacatcccacaatgcttttctttattttcctataagtatctccacgaaatgaactgtgtgaagctgatggaaactgcatttgtttttttcaaagtgttcaaattatggtaggctactgactgtctttttaggagttctaagcttgcctttttaataatttaatttggtttcctttgctcaactctcttatgtaatatcacactgtctgtgagttactcttcttgttctct

SEQ ID NO: 33 - protein, 480 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTELVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVNAWTSGHNDQSQWLQVDLLVPTKVTGIITQGAKDFGHVQFVGSYKLAYSNDGEHWMVHQDEKQRKDKVFQGNFDNDTHRKNVIDPPIYARFIRILPWSWYGRITLRSELLGCAEEE

SEQ ID NO: 34 - DNA, 105 bp, Mus musculus
  CDS (1)..(105)
tgtgaagctgagccttgcagaaatggcggaatatgtaccgaacttgttgctaactactcttgtgaatgcccaggagaatttatgggacgaaattgtcaatataaa

SEQ ID NO: 35 - protein, 35 aa, Mus musculus
CEAEPCRNGGICTELVANYSCECPGEFMGRNCQYK

SEQ ID NO: 36 - DNA, 105 bp, Mus musculus
  CDS (1)..(105)
tgtgaagctgagccttgcagaaatggcggaatatgtaccaaccttgttgctaactactcttgtgaatgcccaggagaatttatgggacgaaattgtcaatataaa

SEQ ID NO: 37 - protein, 35 aa, Mus musculus
CEAEPCRNGGICTNLVANYSCECPGEFMGRNCQYK

SEQ ID NO: 38 - DNA, 105 bp, Mus musculus
  CDS (1)..(105)
tgtgaagctgagccttgcagaaatggcggaatatgtaccgaccttgttgctaacttctcttgtgaatgcccaggagaatttatgggacgaaattgtcaatataaa

SEQ ID NO: 39 - protein, 35 aa, Mus musculus
CEAEPCRNGGICTDLVANFSCECPGEFMGRNCQYK

SEQ ID NO: 40 - protein, 12 aa, Artificial Sequence
  Synthetic peptide
CXXXXXXXXCXC

SEQ ID NO: 41 - DNA, 591 bp, Mus musculus
  CDS (1)..(591)
tgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgt

SEQ ID NO: 42 - protein, 197 aa, Mus musculus
CEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGC

SEQ ID NO: 43 - DNA, 1937 bp, Mus musculus
  CDS (211)..(1050)
tgaggcttctcagcttcagatgcaagtgagtgggtgtctcacagagaagcaaagagaagagaacaggagaaaggtgtttcccttgactgcggaaactttataaagaaaacttagcttctctggagcagtcagcgtcagagttctgtccttgacacctgagtctcctccacaaggctgtgagaaggaaaccctttcctggggctgggtgccatgcagcagcccatgaattacccatgtccccagatcttctgggtagacagcagtgccacttcatcttgggctcctccagggtcagtttttccctgtccatcttgtgggcctagagggccggaccaaaggagaccgccacctccaccaccacctgtgtcaccactaccaccgccatcacaaccactcccactgccgccactgacccctctaaagaagaaggaccacaacacaaatctgtggctaccggtggtatttttcatggttctggtggctctggttggaatgggattaggaatgtatcagctcttccacctgcagaaggaactggcagaactccgtgagttcaccaaccaaagccttaaagtatcatcttttgaaaagcaaatagccaaccccagtacaccctctgaaaaaaaagagccgaggagtgtggcccatttaacagggaacccccactcaaggtccatccctctggaatgggaagacacatatggaaccgctctgatctctggagtgaagtataagaaaggtggccttgtgatcaacgaaactgggttgtacttcgtgtattccaaagtatacttccggggtcagtcttgcaacaaccagcccctaaaccacaaggtctatatgaggaactctaagtatcctgaggatctggtgctaatggaggagaagaggttgaactactgcactactggacagatatgggcccacagcagctacctgggggcagtattcaatcttaccagtgctgaccatttatatgtcaacatatctcaactctctctgatcaattttgaggaatctaagacctttttcggcttgtataagctttaaaagaaaaagcattttaaaatgatctactattctttatcatgggcaccaggaatattgtcttgaatgagagtcttcttaagacctattgagattaattaagactacatgagccacaaagacctcatgaccgcaaggtccaacaggtcagctatccttcattttctcgaggtccatggagtggtccttaatgcctgcatcatgagccagatggaaggaggtctgtgactgagggacataaagctttgggctgctgtgtgacaatgcagaggcacagagaaagaactgtctgatgttaaatggccaagagaattttaaccattgaagaagacacctttacactcacttccagggtgggtctacttactacctcacagaggccgtttttgagacatagttgtggtatgaatatacaagggtgagaaaggaggctcatttgactgataagctagagactgaaaaaaagacagtgtctcattggcaccatctttactgttacctaatgttttctgagccgacctttgatcctaacggagaagtaagagggatgtttgaggcacaaatcattctctacatagcatgcatacctccagtgcaatgatgtctgtgtgtttgtatgtatgagagcaaacagattctaaggagtcatataaataaaatatgtacattatggagtacatattagaaacctgttacatttgatgctagatatctgaatgtttcttggcaataaactctaatagtcttcaaaatcttttattatcagctactgatgctgtttttctttaatacaactagtatttatgctctgaacatcctaatgaggaaaagacaaataaaattatgttatagaatacagaaatgccttaaggacatagactttggaaatc

SEQ ID NO: 44 - protein, 279 aa, Mus musculus
MQQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSYLGAVFNLTSADHLYVNISQLSLINFEESKTFFGLYKL